(19) United States
(12) Patent Application Publication
(10) Pub. No.: US 2019/0333274 A1
(43) Pub. Date: Oct. 31, 2019

(54) Augmented Reality Presentation Associated with a Patient's Medical Condition and/or Treatment

(71) Applicant: International Business Machines Corporation, Armonk, NY (US)

(72) Inventors: Eric W. Brown, New Fairfield, CT (US); Maria Eleftheriou, Mount Kisco, NY (US); Anca Sailer, Scarsdale, NY (US); Ching-Huei Tsou, Briarcliff Manor, NY (US)

(21) Appl. No.: 15/964,687

(22) Filed: Apr. 27, 2018

(51) Int. Cl. G06T 19/00; G16H 10/60; A61B 90/50

(52) U.S. Cl. CPC G06T 19/006; G16H 80/00; A61B 90/50; G16H 10/60

(57) ABSTRACT

A mechanism is provided for implementing an augmented reality display via a head mounted display (HMD) system that indicates areas of a patient's body corresponding to a medical condition and/or treatment of the patient overlayed on the actual view of the patient. A real-time image of an area of a patient's body being viewed by a medical professional is captured via the HMD system. One or more body parts of the patient are identified within the real-time image. The one or more identified body parts are correlated with the patient's electronic medical records (EMRs) indicating the medical condition and/or treatments associated with the patient. An augmented reality display is then generated in the HMD system of one or more areas of the patient's body corresponding to the medical condition and/or treatment of the patient overlaying the real-time image of the area of the patient's body.

### BACKGROUND

The present application relates generally to an improved data processing apparatus and method and more specifically to mechanisms for presenting an augmented reality representation to a medical professional associated with a patient's medical condition and/or treatment.

An electronic health record (EHR) or electronic medical record (EMR) is the systematized collection of patient and population electronically-stored health information in a digital format. These records can be shared across different health care settings. Records are shared through network-connected, enterprise-wide information systems or other information networks and exchanges. EMRs may include a range of data, including demographics, medical history, medication and allergies, immunization status, laboratory test results, radiology images, vital signs, personal statistics like age and weight, and billing information.

EMR systems are designed to store data accurately and to capture the state of a patient across time. It eliminates the need to track down a patient's previous paper medical records and assists in ensuring data is accurate and legible. It can reduce risk of data replication as there is only one modifiable file, which means the file is more likely up to date, and decreases risk of lost paperwork. Due to the digital information being searchable and in a single file, EMRs are more effective when extracting medical data for the examination of possible trends and long term changes in a patient. Population-based studies of medical records may also be facilitated by the widespread adoption of EMRs.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method, in a data processing system, is provided for implementing an augmented reality display via a head mounted display (HMD) system that indicates areas of a patient's body corresponding to a medical condition and/or treatment of the patient overlayed on the actual view of the patient. The illustrative embodiment captures, by a capturing mechanism of a cognitive healthcare system, a real-time image of an area of a patient's body being viewed by a medical professional via the HMD system. The illustrative embodiment identifies one or more body parts of the patient within the real-time image. The illustrative embodiment correlates the one or more identified body parts with the patient's electronic medical records (EMRs) indicating the medical condition and/or treatments associated with the patient. The illustrative embodiment generates an augmented reality display, in the HMD system, of one or more areas of the patient's body corresponding to the medical condition and/or treatment of the patient overlaying the real-time image of the area of the patient's body.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE SEVERAL VIEWS OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive system in a computer network;

FIG. 2 is a block diagram of an example data processing system in which aspects of the illustrative embodiments are implemented;

FIG. 3 illustrates a cognitive system processing pipeline for processing input to generate an overlay in an augmented reality display of a head mounted display (HMD) system in accordance with one illustrative embodiment; and

FIG. 4 depicts an exemplary flowchart of the operation performed by a cognitive healthcare system in implementing an augmented reality display via a head mounted display (HMD) system that indicates the areas of a patient's body corresponding to a medical condition and/or treatment of the patient overlayed on the actual view of the patient captured by the medical professional's eyes in accordance with an illustrative embodiment.

### DETAILED DESCRIPTION

In current medial assessments, medical professionals must sift through a large amount of medical information about a patient to attempt to find the most relevant information for treating the patient. Typically, these assessments requires that the medical professional have access to a physical medical file for the patient, or with the increased use of electronic medical records (EMR), parse through large amounts of data represented in the patient's EMR. This requires a large amount of time an effort on the part of medical professionals who already have limited time to treat patients. Because of this, patients often feel as if the medical professional does not know them personally or treat them on a personal basis as well as not spending enough time with the patient during a scheduled appointment, i.e. the medical professional is too busy searching through patient EMR data or physical files to get an idea of how to treat the patient rather than actually interacting with the patient and maintaining eye contact with the patient.

Accordingly, the illustrative embodiments provide mechanisms for implementing an augmented reality display via a head mounted display (HMD) system, such as via a worn headset, glasses, or the like, that indicates the areas of a patient's body corresponding to a medical condition and/or treatment of the patient overlayed on the actual view of the patient captured by the medical professional's eyes. The mechanisms of the invention capture images of the area of the patient's body being viewed by the medical professional. Based on the part of the patient's body being viewed, the mechanisms identify the corresponding body parts in the view and correlate those body parts with the patient's electronic medical record (EMR) data indicating the medical condition and/or treatments associated with the patient. In some cases, facial recognition may be utilized to identify the particular patient being viewed.

The superimposed graphical representations on the patient's body may be medical condition and implementation specific. That is, the superimposed graphical representations may include graphical images representing medical conditions, highlighting of portions of the body affected or needing to be further investigated, textual data representing lab results, treatment options, medical codes, or the like. The mechanisms also provide access to a medical corpus of data annotated for multiple media views to allow the real time selection of media suitable for a given patient mood, the time of the day, the medical professional's schedule availability, or the like. With regard to the medical professional's schedule, depending on the schedule, a more compressed (basic organ model) may be displayed with the availability is limited, whereas a detailed (surgery technique simulated on the patient organ) may be displayed when the availability is extended.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

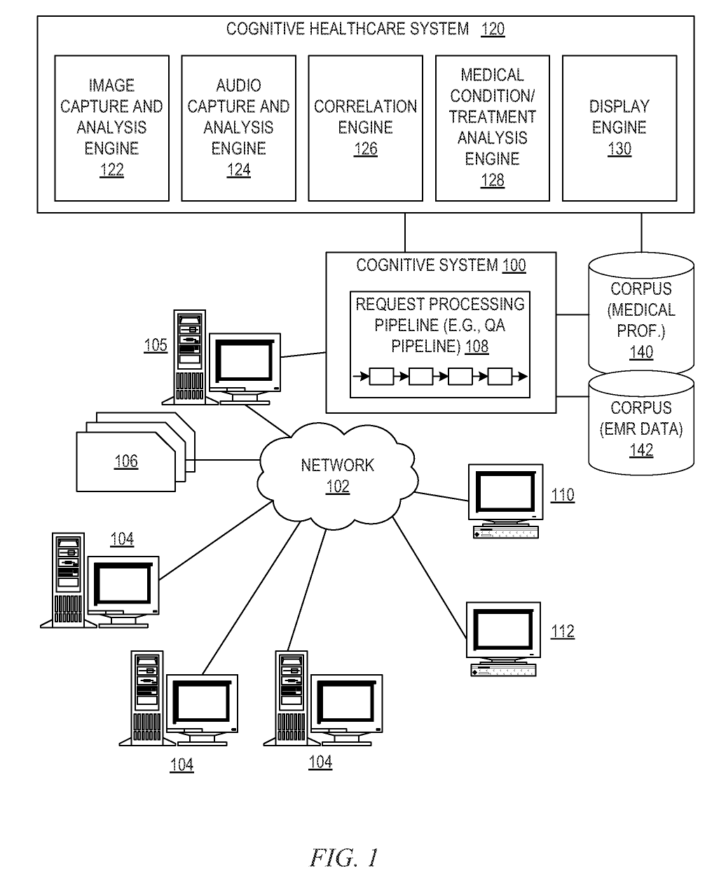

As noted above, the present invention provides mechanisms for the illustrative embodiments provide mechanisms for implementing an augmented reality display via a head mounted display (HMD) system, such as via a worn headset, glasses, or the like, that indicates the areas of a patient's body corresponding to a medical condition and/or treatment of the patient overlayed on the actual view of the patient captured by the medical professional's eye. The illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for implementing an augmented reality display via a head mounted display (HMD) system, such as via a worn headset, glasses, or the like, that indicates the areas of a patient's body corresponding to a medical condition and/or treatment of the patient overlayed on the actual view of the patient captured by the medical professional's eye. Accordingly, in order to identify the medical condition and/or treatment associated with the patient, the cognitive system implements a request processing pipeline, request processing methodology, and request processing computer program product with which the mechanisms of the illustrative embodiments are implemented. These requests may be provided as structure or unstructured request messages, natural language questions, or any other suitable format for requesting an operation to be performed by the cognitive system. As described in more detail hereafter, the particular application that is implemented in the cognitive system of the present invention is an application for implementing an augmented reality display via a head mounted display (HMD) system that indicates the areas of a patient's body corresponding to a medical condition and/or treatment of the patient overlayed on the actual view of the patient captured by the medical professional's eye.

It should be appreciated that the cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests (or questions in implementations using a QA pipeline), depending on the desired implementation. For example, in some cases, a first request processing pipeline may be trained to operate on input requests directed to identifying a medical condition of the patient such that the patient's doctor may see the area of the patient to which the medical condition is associated. In other cases, for example, the request processing pipelines may be configured to provide different types of cognitive functions or support different types of applications, such as one request processing pipeline being used for identifying a medical treatment of the patient such that a nurse who is treating the patient may see the area of the patient to which the a treatment to be applied is associated, etc.

Moreover, each request processing pipeline may have its associated corpus or corpora that they ingest and operate on, e.g., one corpus for medical conditions documents and another corpus for medical treatments related documents in the above examples. In some cases, the request processing pipelines may each operate on the same domain of input questions but may have different configurations, e.g., different annotators or differently trained annotators, such that different analysis and potential answers are generated. The cognitive system may provide additional logic for routing input questions to the appropriate request processing pipeline, such as based on a determined domain of the input request, combining and evaluating final results generated by the processing performed by multiple request processing pipelines, and other control and interaction logic that facilitates the utilization of multiple request processing pipelines.

It should be appreciated that while the present invention will be described in the context of the cognitive system implementing one or more request pipelines that operate on a request, the illustrative embodiments are not limited to such. Rather, the mechanisms of the illustrative embodiments may operate on requests that are not posed as “questions” but are formatted as requests for the cognitive system to perform cognitive operations on a specified set of input data using the associated corpus or corpora and the specific configuration information used to configure the cognitive system.

As will be discussed in greater detail hereafter, the illustrative embodiments may be integrated in, augment, and extend the functionality of the request processing pipeline with regard to implementing an augmented reality display that indicates the areas of a patient's body corresponding to a medical condition and/or treatment of the patient overlayed on the actual view of the patient captured by the medical professional's eyes. For example, if a patient has a medical condition of appendicitis, then when the doctor view's the patient's body through the augmented reality display of a mounted display (HMD) system, the patient's appendix will be shown overlaying the lower abdomen of the patient's body when the lower abdomen of the patient's body is viewed through the augmented reality display of the HMD system.

It should be appreciated that the mechanisms described in FIGS. 1-3 are only examples and are not intended to state or imply any limitation with regard to the type of cognitive system mechanisms with which the illustrative embodiments are implemented. Many modifications to the example cognitive system shown in FIGS. 1-3 may be implemented in various embodiments of the present invention without departing from the spirit and scope of the present invention.

As an overview, a cognitive system is a specialized computer system, or set of computer systems, configured with hardware and/or software logic (in combination with hardware logic upon which the software executes) to emulate human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that approximate a human thought process as well as enable people and machines to interact in a more natural manner so as to extend and magnify human expertise and cognition. A cognitive system comprises artificial intelligence logic, such as natural language processing (NLP) based logic, for example, and machine learning logic, which may be provided as specialized hardware, software executed on hardware, or any combination of specialized hardware and software executed on hardware. The logic of the cognitive system implements the cognitive operation(s), examples of which include, but are not limited to, question answering, identification of related concepts within different portions of content in a corpus, intelligent search algorithms, such as Internet web page searches, for example, medical diagnostic and treatment recommendations, and other types of recommendation generation, e.g., items of interest to a particular user, potential new contact recommendations, or the like.

IBM Watson™ is an example of one such cognitive system which can process human readable language and identify inferences between text passages with human-like high accuracy at speeds far faster than human beings and on a larger scale. In general, such cognitive systems are able to perform the following functions:
Navigate the complexities of human language and understanding
Ingest and process vast amounts of structured and unstructured data
Generate and evaluate hypothesis
Weigh and evaluate responses that are based only on relevant evidence
Provide situation-specific advice, insights, and guidance
Improve knowledge and learn with each iteration and interaction through machine learning processes
Enable decision making at the point of impact (contextual guidance)
Scale in proportion to the task
Extend and magnify human expertise and cognition
Identify resonating, human-like attributes and traits from natural language
Deduce various language specific or agnostic attributes from natural language
High degree of relevant recollection from data points (images, text, voice) (memorization and recall)
Predict and sense with situational awareness that mimic human cognition based on experiences
Answer questions based on natural language and specific evidence

In one aspect, cognitive systems provide mechanisms for responding to requests posed to these cognitive systems using a request processing pipeline and/or process requests which may or may not be posed as natural language requests. The requests processing pipeline is an artificial intelligence application executing on data processing hardware that responds to requests pertaining to a given subject-matter domain presented in natural language. The request processing pipeline receives inputs from various sources including input over a network, a corpus of electronic documents or other data, data from a content creator, information from one or more content users, and other such inputs from other possible sources of input. Data storage devices store the corpus of data. A content creator creates content in a document for use as part of a corpus of data with the request processing pipeline. The document may include any file, text, article, or source of data for use in the requests processing system. For example, a request processing pipeline accesses a body of knowledge about the domain, or subject matter area, e.g., financial domain, medical domain, legal domain, etc., where the body of knowledge (knowledgebase) can be organized in a variety of configurations, e.g., a structured repository of domain-specific information, such as ontologies, or unstructured data related to the domain, or a collection of natural language documents about the domain.

Content users input requests to cognitive system which implements the request processing pipeline. The request processing pipeline then responds to the requests using the content in the corpus of data by evaluating documents, sections of documents, portions of data in the corpus, or the like. When a process evaluates a given section of a document for semantic content, the process can use a variety of conventions to query such document from the request processing pipeline, e.g., sending the query to the request processing pipeline as a well-formed requests which is then interpreted by the request processing pipeline and a response is provided containing one or more responses to the request. Semantic content is content based on the relation between signifiers, such as words, phrases, signs, and symbols, and what they stand for, their denotation, or connotation. In other words, semantic content is content that interprets an expression, such as by using Natural Language Processing.

As will be described in greater detail hereafter, the request processing pipeline receives a request, parses the request to extract the major features of the request, uses the extracted features to formulate queries, and then applies those queries to the corpus of data. Based on the application of the queries to the corpus of data, the request processing pipeline generates a set of responses to the request, by looking across the corpus of data for portions of the corpus of data that have some potential for containing a valuable response to the request. The request processing pipeline then performs deep analysis on the language of the request and the language used in each of the portions of the corpus of data found during the application of the queries using a variety of reasoning algorithms. There may be hundreds or even thousands of reasoning algorithms applied, each of which performs different analysis, e.g., comparisons, natural language analysis, lexical analysis, or the like, and generates a score. For example, some reasoning algorithms may look at the matching of terms and synonyms within the language of the request and the found portions of the corpus of data. Other reasoning algorithms may look at temporal or spatial features in the language, while others may evaluate the source of the portion of the corpus of data and evaluate its veracity.

As mentioned above, request processing pipeline mechanisms operate by accessing information from a corpus of data or information (also referred to as a corpus of content), analyzing it, and then generating answer results based on the analysis of this data. Accessing information from a corpus of data typically includes: a database query that answers requests about what is in a collection of structured records, and a search that delivers a collection of document links in response to a query against a collection of unstructured data (text, markup language, etc.). Conventional request processing systems are capable of generating answers based on the corpus of data and the input request, verifying answers to a collection of request for the corpus of data, correcting errors in digital text using a corpus of data, and selecting responses to requests from a pool of potential answers, i.e. candidate answers.

FIG. 1 depicts a schematic diagram of one illustrative embodiment of a cognitive system 100 implementing a request processing pipeline 108, which in some embodiments may be a request processing pipeline, in a computer network 102. For purposes of the present description, it will be assumed that the request processing pipeline 108 that operates on structured and/or unstructured requests in the form of input questions. One example of a question processing operation which may be used in conjunction with the principles described herein is described in U.S. Patent Application Publication No. 2011/0125734, which is herein incorporated by reference in its entirety. The cognitive system 100 is implemented on one or more computing devices 104A-D (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-D. The network 102 includes multiple computing devices 104A-D, which may operate as server computing devices, and 110-112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 enables request processing functionality for one or more cognitive system users via their respective computing devices 110-112. In other embodiments, the cognitive system 100 and network 102 may provide other types of cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of a natural language question, natural language request for information, natural language request for the performance of a cognitive operation, or the like. For example, the cognitive system 100 receives input from the network 102, a corpus or corpora of electronic documents 106, cognitive system users, and/or other data and other possible sources of input. In one embodiment, some or all of the inputs to the cognitive system 100 are routed through the network 102. The various computing devices 104A-D on the network 102 include access points for content creators and cognitive system users. Some of the computing devices 104A-D include devices for a database storing the corpus or corpora of data 106 (which is shown as a separate entity in FIG. 1 for illustrative purposes only). Portions of the corpus or corpora of data 106 may also be provided on one or more other network attached storage devices, in one or more databases, or other computing devices not explicitly shown in FIG. 1. The network 102 includes local network connections and remote connections in various embodiments, such that the cognitive system 100 may operate in environments of any size, including local and global, e.g., the Internet.

In one embodiment, the content creator creates content in a document of the corpus or corpora of data 106 for use as part of a corpus of data with the cognitive system 100. The document includes any file, text, article, or source of data for use in the cognitive system 100. Cognitive system users access the cognitive system 100 via a network connection or an Internet connection to the network 102, and input questions/requests to the cognitive system 100 that are answered/processed based on the content in the corpus or corpora of data 106. In one embodiment, the questions/requests are formed using natural language. The cognitive system 100 parses and interprets the question/request via request processing pipeline 108, and provides a response to the cognitive system user, e.g., cognitive system user 110, containing one or more answers to the question posed, response to the request, results of processing the request, or the like. In some embodiments, the cognitive system 100 provides a response to users in a ranked list of candidate answers/responses while in other illustrative embodiments, the cognitive system 100 provides a single final answer/response or a combination of a final answer/response and ranked listing of other candidate answers/responses.

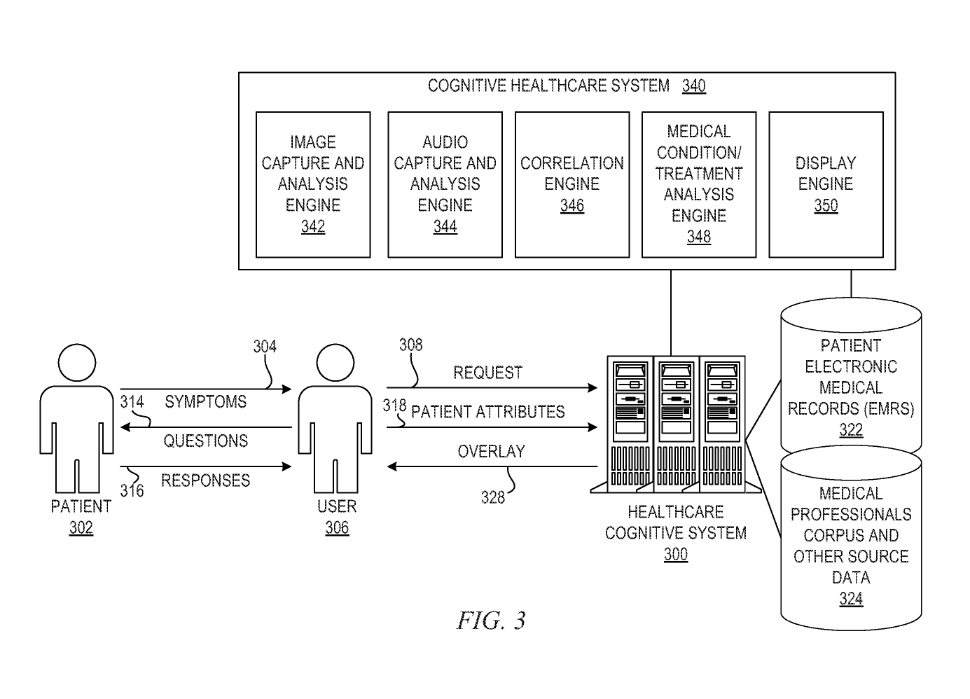

The cognitive system 100 implements the request processing 108 which comprises a plurality of stages for processing an input question/request based on information obtained from the corpus or corpora of data 106. The request processing 108 generates answers/responses for the input question or request based on the processing of the input question/request and the corpus or corpora of data 106. The request processing 108 will be described in greater detail hereafter with regard to FIG. 3.

In some illustrative embodiments, the cognitive system 100 may be the IBM Watson™ cognitive system available from International Business Machines Corporation of Armonk, N.Y., which is augmented with the mechanisms of the illustrative embodiments described hereafter. As outlined previously, a pipeline of the IBM Watson™ cognitive system receives an input question or request which it then parses to extract the major features of the question/request, which in turn are then used to formulate queries that are applied to the corpus or corpora of data 106. Based on the application of the queries to the corpus or corpora of data 106, a set of hypotheses, or candidate answers/responses to the input question/request, are generated by looking across the corpus or corpora of data 106 for portions of the corpus or corpora of data 106 (hereafter referred to simply as the corpus 106) that have some potential for containing a valuable response to the input question/response (hereafter assumed to be an input question). The request processing 108 of the IBM Watson™ cognitive system then performs deep analysis on the language of the input question and the language used in each of the portions of the corpus 106 found during the application of the queries using a variety of reasoning algorithms.

The scores obtained from the various reasoning algorithms are then weighted against a statistical model that summarizes a level of confidence that the request processing 108 of the IBM Watson™ cognitive system 100, in this example, has regarding the evidence that the potential candidate answer is inferred by the question. This process is be repeated for each of the candidate answers to generate ranked listing of candidate answers which may then be presented to the user that submitted the input question, e.g., a user of client computing device 110, or from which a final answer is selected and presented to the user. More information about the request processing 108 of the IBM Watson™ cognitive system 100 may be obtained, for example, from the IBM Corporation website, IBM Redbooks, and the like. For example, information about the pipeline of the IBM Watson™ cognitive system can be found in Yuan et al., “Watson and Healthcare,” IBM developerWorks, 2011 and “The Era of Cognitive Systems: An Inside Look at IBM Watson and How it Works” by Rob High, IBM Redbooks, 2012.

As noted above, while the input to the cognitive system 100 from a client device may be posed in the form of a natural language question, the illustrative embodiments are not limited to such. Rather, the input question may in fact be formatted or structured as any suitable type of request which may be parsed and analyzed using structured and/or unstructured input analysis, including but not limited to the natural language parsing and analysis mechanisms of a cognitive system such as IBM Watson™, to determine the basis upon which to perform cognitive analysis and providing a result of the cognitive analysis. In the case of a healthcare based cognitive system, this analysis may involve processing patient's electronic medical records, medical guidance documentation from one or more corpora, and the like, to provide a healthcare oriented cognitive system result.

In the context of the present invention, cognitive system 100 may provide a cognitive functionality for implementing an augmented reality display via a head mounted display (HMD) system that indicates the areas of a patient's body corresponding to a medical condition and/or treatment of the patient overlayed on the actual view of the patient captured by the medical professional's eye. For example, depending upon the particular implementation, the healthcare based operations may comprise patient diagnostics, medical practice management systems, personal patient care plan monitoring, patient's electronic medical record (EMR) evaluation for various purposes, such as for identifying a medical condition and/or treatment of a patient and implementing an augmented reality display that indicates the areas of a patient's body corresponding to the medical condition and/or treatment of the patient overlayed on the actual view of the patient captured by the medical professional's eye. Thus, the cognitive system 100 may be a healthcare cognitive system 100 that operates in the medical or healthcare type domains and which may process requests for such healthcare operations via the request processing pipeline 108 input as either structured or unstructured requests, natural language input questions, or the like. In one illustrative embodiment, the cognitive system 100 is a cognitive healthcare system 100 that analyzes a patient's EMR and provides an indication of the patient's medical condition and/or treatment that the patient is receiving. Utilizing the identified medical condition and/or treatment, the cognitive healthcare system 100 isolates the particular portion(s) of the patient's body associated with the particular medical condition and/or treatment and implements an augmented reality display that indicates the areas of a patient's body corresponding to the medical condition and/or treatment of the patient overlayed on the actual view of the patient's body captured by the medical professional's eye.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for implementing a cognitive healthcare system 120 that indicates an area of a patient's body corresponding to a medical condition and/or treatment of the patient in an augmented reality display overlayed on the actual view of the patient captured by the medical professional's eye. As shown in FIG. 1, cognitive healthcare system 120 comprises image capture and analysis engine 122, audio capture and analysis engine 124, correlation engine 126, medical condition/treatment analysis engine 128, and display engine 130.

In cognitive system 100 and, more specifically, cognitive healthcare system 120, image capture and analysis engine 122 captures one or more real-time images of a patient that is being cared for by a medical professional and/or the medical professional. That is, image capture and analysis engine 122 may utilize one or more cameras associated with the HMD system, such as cameras facing the patient, retinal cameras pointed at the medical professional's eyes, or the like, to capture one or more images. Image capture and analysis engine 122 utilizes the one or more real-time images for numerous different aspects of the illustrative embodiments. In one embodiment, image capture and analysis engine 122 utilizes images of the medical professional's eyes to identify the medical professional that is caring for the patient. In another embodiment, image capture and analysis engine 122 captures an image of the patient's face that may be used to identify which patient is being seen by the medical professional and/or a mood of the patient. That is, image capture and analysis engine 122 may capture an image of the patient's face that may be used in identifying the patient that is being cared for through facial recognition. Further, image capture and analysis engine 122 may capture an image of the patient's face that may be used in identifying a mood of the patient by identifying whether the patient is crying or whether the facial expressions denote fear, happiness, concern, worry, or the like.

Also, in cognitive healthcare system 120, audio capture and analysis engine 124 captures one or more audible utterances by the patient and/or the medical professional that is caring for the patient. Audio capture and analysis engine 124 utilizes the one or more audible utterances for numerous different aspects of the illustrative embodiments. In one embodiment, audio capture and analysis engine 124 captures an audible utterance by the medical professional which may be used to identify the medical professional that is caring for the patient. In another embodiment, audio capture and analysis engine 124 captures an audible instruction provided by the medical professional that may be used by cognitive healthcare system 120 in presenting further information to the medical professional via the augmented reality display of the HMD system. In still another embodiment, audio capture and analysis engine 124 captures an audible utterance of the patient to identify a mood of the patient. That is, audio capture and analysis engine 124 may capture sounds of a patient crying, trepidation in the patient's voice, laughter, or the like, used to identify one or more of concern, worry, fear, happiness, or the like.

Utilizing the one or more images captured by image capture and analysis engine 122 and the one or more audible utterances captured by audio capture and analysis engine 124, correlation engine 126 performs numerous correlations in order to provide necessary information to the medical professional. One exemplary correlation is to identify the medical professional that is caring for the patient where correlation engine 126 utilizes facial recognition to compare the one or more images to images of medical professionals stored in medical professional corpus or corpora of data 140. Another exemplary correlation is to identify the medical professional that is caring for the patient where correlation engine 126 utilizes voice recognition to compare the one or more audible utterances to voice patterns of medical professionals stored in medical professional corpus or corpora of data 140. Similarly, correlation engine 126 performs a correlation to identify the patient that is cared for by the medical professional. To identify the patient, correlation engine 126 utilizes facial recognition to compare the one or more images to images within a set of electronic medical records (EMRs) for patients stored in corpus or corpora of data 142. In addition to or as a completely different form of identification, correlation engine 126 utilizes voice recognition to compare the one or more audible utterances to voice patterns of patients within a set of electronic medical records (EMRs) for patients stored in corpus or corpora of data 142.

In addition to identifying the medical professional and the patient, correlation engine 126 also identifies one or more body parts of the patient that is being viewed by the medical professional. The identification of the particular body part(s) that are being viewed is particularly important when overlaying a medical condition of the patient on the actual view of the patient captured by the medical professional's eyes through the augmented reality display. That is, based on the part of the patient's body being viewed by the medical professional, correlation engine 126 identifies the particular body part(s) for further correlation to those body parts in the patient's electronic medical record (EMR) data indicating the medical condition and/or treatments associated with the patient. Thus, as correlation engine 126 identifies the particular body part(s) that are being viewed, medical condition/treatment analysis engine 128 analyzes the electronic medical records (EMR) of the patient stored in corpus or corpora of data 142 to identify a medical condition and/or treatment associated with the patient. Utilizing the identified medical condition and/or treatment of the patient, correlation engine 126 identifies a portion of the patient's body that is associated with the particular medical condition and/or treatments as that part of the patient's body is viewed by the medical professional. For example, if a patient has a medical condition of appendicitis, then when the medical professional views the patient's body through the augmented reality display and the patient's lower abdomen comes into view, correlation engine 126 will correlate the view of the patient's lower abdomen with the medical condition of the patient and provide an overlay of an appendix to be shown overlaying the lower abdomen of the patient's body. Correlation engine 126 provides this overlay to display engine 130 and display engine 130 presents the overlay to the medical professional via the augmented reality display in the HMD system.

Of particular note to the illustrative embodiments, is that the overlay provided by correlation engine 126 may be varied depending on the view that the medical professional needs. That is, if the medical professional is a nurse, then correlation engine 126 may provide a basic organ model showing a generic organ. However, if the medical professional is a doctor, then correlation engine 126 may provide an actual x-ray overlay of the organ. Still further, if the medical professional is a surgeon, then correlation engine 126 may provide a computerized axial tomography (CAT) scan (CT) overlay or a magnetic resonance imaging (MRI) scan overlay of the entire area. In addition to providing a basic organ model, an x-ray, CT scan, MRI scan, or the like, correlation engine 126 may also provide one or more of dissection models; overlapping organ systems; x-rays, CT scans, MRI scans, or the like, from previous medical condition/treatments; points of surgery or pressure; or the like. An indication of any additional information to provide may be identified by monitoring eye moves, facial expressions, head moved, audible utterances, or the like from the medical professional via image capture and analysis engine 122 and/or audio capture and analysis engine 124.

Additionally, the overlay provided by correlation engine 126 may be based on the time that the medical professional has to spend with the patient. For example, based on the medical professional's schedule, which may be accessed by correlation engine 126 through medical professional corpus or corpora of data 140, the medical professional may only have a few minutes to spend with the patient as may occur during morning rounds. Thus, correlation engine 126 may provide a basic organ model showing a generic organ. However, if the schedule shows that the medical professional is performing a surgical consult prior to a surgery, then correlation engine 126 may provide an x-ray overlay, a computerized axial tomography (CAT) scan (CT) overlay, or a magnetic resonance imaging (MRI) scan overlay of the entire area. Further, whether or not the schedule shows to permits more time, if the medical professional requests, correlation engine 126 may provide any additionally overlays. That is, even though the medical professional's schedule indicates that the medical professional may only have a few minutes to spend with the patient as may occur during morning rounds and initially provide a basic organ model showing a generic organ, if the medical professional requests additional information, then correlation engine 126 may provide an x-ray overlay; a CT scan overlay; a MRI scan overlay; one or more of dissection models; overlapping organ systems; x-rays, CT scans, MRI scans, or the like, from previous medical condition/treatments; points of surgery or pressure, or the like. An indication of any additional information to provide may be identified by monitoring eye moves, facial expressions, head moved, audible utterances, or the like from the medical professional via image capture and analysis engine 122 and/or audio capture and analysis engine 124.

Still further, correlation engine 126 may provide an overlay that is based on the particular specialty of the medical professional that is caring for the patient. That is, if the identity of the medical professional is an anesthesiologist or anesthetist, then correlation engine 126 may provide an organ overlay that is not even associated with the particular medical condition. That is, an anesthesiologist or anesthetist may be more concerned with the patient's lungs, airways, nasal cavities, or the like. Conversely, if the identity of the medical professional is a surgeon, then correlation engine 126 may provide an organ overlay that is directly related to the particular medical condition. Further, if the identity of the medical professional is a nurse who is providing medications to the patient, then correlation engine 126 may provide an organ overlay of where the medication is to be administered, such as a particular arm, area of an arm, or the like.

In addition to providing an overlay that identifies graphical images representing medical conditions, highlighting of portions of the body affected or needing to be further investigated, correlation engine 126 may also provide textual data representing lab results, treatment options, medical codes, latest medical research studies, available organs for transplant, or the like. An indication of any additional information to provide may be identified by monitoring eye moves, facial expressions, head moved, audible utterances, or the like from the medical professional. That is, based on inputs provided by the medical professional, correlation engine may identify the requested textual data, which display engine 130 then displays on the augmented reality display of the HMD system. Still further, based on a mood identified using the one or more images and/or the one or more audible utterances of the patient, correlation engine 126 may provide an indication of how the medical professional should be presenting information to the patient. That is, if the patient is identified as calm, then correlation engine 126 may provide an indication to the medical professional to speak in a relaxed tone. However, if the patient is identified as nervous, then the medical then correlation engine 126 may provide an indication to the medical professional to use more reassuring tones.

Additionally, once a medical professional selects, indicates, or otherwise identifies a treatment that is to be followed for the patient, which may be identified by monitoring eye moves, facial expressions, head moved, audible utterances, or the like from the medical professional, correlation engine 126 may notify one or more other medical professionals of the treatment through one or more electronic notification means, which may include scheduling a surgery, instruments to be provided during surgery, requests for a consultation, medications to be administered, or the like, in real-time, near real-time, or non-real-time. An indication of the treatment may be identified by monitoring eye moves, facial expressions, head moved, audible utterances, or the like from the medical professional via image capture and analysis engine 122 and/or audio capture and analysis engine 124.

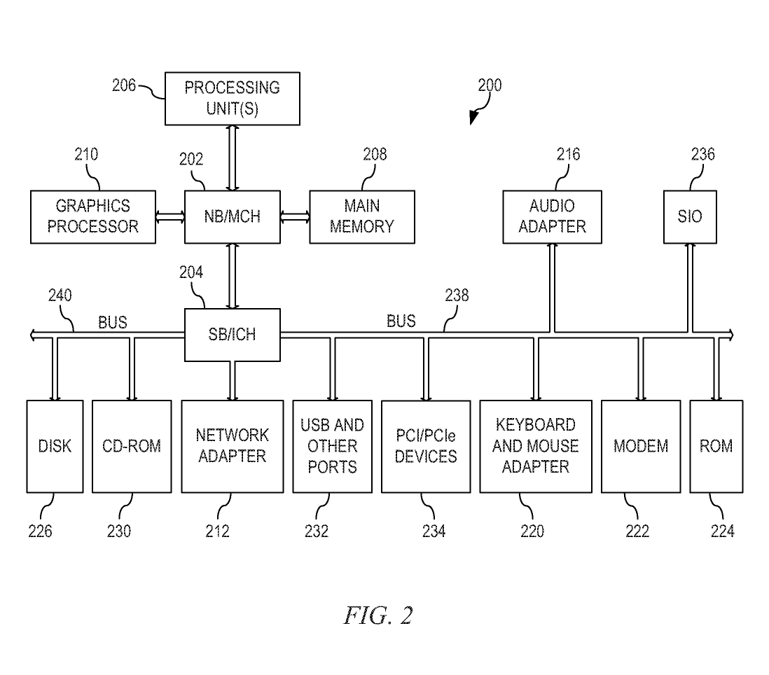

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which, which implements a cognitive system 100 and request processing pipeline 108 augmented to include the additional mechanisms of the illustrative embodiments described hereafter.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 8®. An object-oriented programming system, such as the Java™ programming system, may run in conjunction with the operating system and provides calls to the operating system from Java™ programs or applications executing on data processing system 200.

As a server, data processing system 200 may be, for example, an IBM® eServe™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

FIG. 3 is an example diagram illustrating an interaction of elements of a cognitive system in accordance with one illustrative embodiment. The example diagram of FIG. 3 depicts an implementation of a cognitive system 300, which may be a cognitive system such as cognitive system 100 described in FIG. 1, that is configured to implement an augmented reality display via a head mounted display (HMD) system, such as via a worn headset, glasses, or the like, that indicates the areas of a patient's body corresponding to a medical condition and/or treatment of the patient overlayed on the actual view of the patient captured by the medical professional's eyes. However, it should be appreciated that this is only an example implementation and other healthcare operations may be implemented in other embodiments of the healthcare cognitive system 300 without departing from the spirit and scope of the present invention.

Moreover, it should be appreciated that while FIG. 3 depicts patient 302 and medical professional 306 as human figures, the interactions with and between these entities may be performed using numerous devices, including, but not limited to, computing devices, medical equipment, and/or the like. For example, interactions 304, 314, 316, and 330 between patient 302 and user 306 may be performed orally, e.g., a doctor interviewing a patient, and may involve the use of one or more medical instruments, monitoring devices, or the like, such as the head mounted display (HMD) system of the illustrative embodiments, to collect information that may be input to the cognitive system 300 as patient attributes 318. Interactions between user 306 and cognitive system 300 will be electronic via a user computing device (not shown), such as a client computing device 110 or 112 in FIG. 1, which is the illustrative embodiments is the HMD system, communicating with cognitive system 300 via one or more data communication links and potentially one or more data networks.

As shown in FIG. 3, in accordance with one illustrative embodiment, a patient 302 presents symptoms 304 of a medical malady or condition to user 306, such as a medical professional, healthcare practitioner, technician, or the like. User 306 may interact with patient 302 via a question 314 and response 316 exchange where user 306 gathers more information about patient 302, symptoms 304, and the medical malady or condition of patient 302. It should be appreciated that the questions/responses may in fact also represent user 306 gathering information from patient 302 using various medical equipment, e.g., the HMD system, blood pressure monitors, thermometers, wearable health and activity monitoring devices associated with patient 302 such as a FitBit™, a wearable heart monitor, or any other medical equipment that may monitor one or more medical characteristics of patient 302. In some cases such medical equipment may be medical equipment typically used in hospitals or medical centers to monitor vital signs and medical conditions of patients that are present in hospital beds for observation or medical treatment. In accordance with the illustrative embodiments, the medical equipment is the HMD system that gathers both images and audible utterances from both patient 202 and user 306.

In response, user 306 submits request 308 to cognitive system 300, such as via the HMD system that is configured to allow users to submit requests to cognitive system 300 in a format that cognitive system 300 is able to parse and process. Request 308 may include, or be accompanied with, information identifying attributes 318 of patient 302 and user 306. That is, the above mentions HMD system may capture one or more real-time images of patient 302 and/or user 306 that is caring for patient 302 as well as one or more audible utterances by patient 302 and/or user 306 that is caring for the patient. Thus, patient attributes 318 may include, for example, an image of the patent's face of an audible utterance from patient 302 from which patient EMRs 322 for patient 302 may be retrieved, demographic information about patient 302, symptoms 304, and other pertinent information obtained from responses 316 to questions 314 or information obtained from medical equipment used to monitor or gather data about the condition of patient 302, including a medical conditions associated with patient 302. Any information about patient 302 that may be relevant to a cognitive evaluation of patient 302 by cognitive system 300 may be included in request 308 and/or patient attributes 318.

Cognitive system 300 is specifically configured to perform an implementation specific healthcare-oriented cognitive operation. In the depicted example, this cognitive precision cohort operation is directed to indicating an area of a patient's body corresponding to a medical condition and/or treatment of the patient in an augmented reality display of the HMD system overlayed on the actual view of the patient captured by the eyes of user 306 to assist user 306 in caring for patient 302 based on their reported symptoms 304 and other information gathered about patient 302 via question 314 and response 316 process and/or medical equipment monitoring/data gathering. Cognitive system 300 operates on request 308 and patient attributes 318 utilizing information gathered from patient EMRs 322 associated with the patient 302 to identify a medical condition of patient 302.

For example, based on request 308 and patient attributes 318, cognitive system 300 may operate on the request to parse request 308 and patient attributes 318 to determine not only which patient is being treated as well as the specific medical condition that patient 302 has as well as any overlays associated with the medical condition that are available for presentation via a display of the HMD system. Thus, cognitive system 300 may operate on the request to parse request 308 and patient attributes 318 to determines what is being requested and the criteria upon which the request is to be generated as identified by patient attributes 318, and may perform various operations for generating queries that are sent to patient EMRs 322 to retrieve data, generate associated indications associated with the data, and provides supporting evidence found in patient EMRs 322. In the depicted example, patient EMRs 322 is a patient information repository that collects patient data from a variety of sources, e.g., hospitals, laboratories, physicians' offices, health insurance companies, pharmacies, etc. Patient EMRs 322 store various information about individual patients, such as patient 302, in a manner (structured, unstructured, or a mix of structured and unstructured formats) that the information may be retrieved and processed by cognitive system 300. This patient information may comprise various demographic information about patients, personal contact information about patients, employment information, health insurance information, laboratory reports, physician reports from office visits, hospital charts, historical information regarding previous diagnoses, symptoms, treatments, prescription information, etc. Based on an identifier of the patient 302, the patient's corresponding EMRs 322 from this patient repository may be retrieved by cognitive system 300 and searched/processed to provide treatment pathways 328 that a similar cohort of patients have followed.

In accordance with the illustrative embodiments herein, cognitive system 300 is augmented to include cognitive healthcare system 340. Cognitive healthcare system 340 comprises image capture and analysis engine 342, audio capture and analysis engine 344, correlation engine 346, medical condition/treatment analysis engine 348, and display engine 350, which operate in a similar manner as previously described above with regard to corresponding elements 122-130 in FIG. 1. That is, image capture and analysis engine 342 captures one or more real-time images of patient 302 and/or user 306 identified from patient attributes 318. Image capture and analysis engine 342 utilizes the one or more real-time images for numerous different aspects of the illustrative embodiments. In one embodiment, image capture and analysis engine 342 utilizes images of the eyes of user 306 to identify the particular medical professional that is caring for patient 320. In another embodiment, image capture and analysis engine 342 captures an image of the face of patient 302 that may be used to identify which patient is being seen by user 306 and/or a mood of patient 302. That is, image capture and analysis engine 342 may capture an image of the face of patient 302 that may be used in identifying the patient that is being cared for through facial recognition. Further, image capture and analysis engine 342 may capture an image of the face of patient 302 that may be used in identifying a mood of the patient by identifying whether patient 302 is crying or whether the facial expressions denote fear, happiness, concern, worry, or the like.

Audio capture and analysis engine 344 captures one or more audible utterances by patient 302 and/or user 306 that is caring for patient 302. Audio capture and analysis engine 344 utilizes the one or more audible utterances for numerous different aspects of the illustrative embodiments. In one embodiment, audio capture and analysis engine 344 captures an audible utterance by user 306 which may be used to identify the medical professional that is caring for patient 302. In another embodiment, audio capture and analysis engine 344 captures an audible instruction provided by user 306 that may be used by cognitive healthcare system 340 in presenting further information to user 306 via the augmented reality display of the HMD system. In still another embodiment, audio capture and analysis engine 344 captures an audible utterance of patient 302 to identify a mood of patient 302. That is, audio capture and analysis engine 344 may capture sounds of a patient crying, trepidation in the patient's voice, laughter, or the like, used to identify one or more of concern, worry, fear, happiness, or the like.

Utilizing the one or more images captured by image capture and analysis engine 342 and the one or more audible utterances captured by audio capture and analysis engine 344, correlation engine 346 performs numerous correlations in order to provide necessary information to the medical professional. One exemplary correlation is to identify user 306 that is caring for the patient where correlation engine 346 utilizes facial recognition to compare the one or more images to images of medical professionals stored in medical professional corpus and other source data 324. Another exemplary correlation is to identify user 306 that is caring for the patient where correlation engine 346 utilizes voice recognition to compare the one or more audible utterances to voice patterns of medical professionals stored in medical professional corpus and other source data 324. Similarly, correlation engine 346 performs a correlation to identify patient 302 that is being cared for by user 306. To identify patient 302, correlation engine 346 may utilize facial recognition to compare the one or more images to images within a set of electronic medical records (EMRs) for patients stored in patient EMRs 322. In addition to or as a completely different form of identification, correlation engine 346 utilizes voice recognition to compare the one or more audible utterances to voice patterns of patients within a set of electronic medical records (EMRs) for patients stored in patient EMRs 322.

In addition to identifying user 306 and patient 302, correlation engine 346 also identifies one or more body parts of patient 302 that is being viewed by user 306 via the HMD system. The identification of the particular body part(s) that are being viewed is particularly important when overlaying a medical condition of patient 302 on the actual view of patient captured by the eyes of user 306 through the augmented reality display of the HMD system. That is, based on the part of body of user 302 being viewed by user 306, correlation engine 346 identifies the particular body part(s) for further correlation to those body parts in the data of patient EMRs 322 indicating the medical condition and/or treatments associated with patient 302. Thus, as correlation engine 346 identifies the particular body part(s) that are being viewed, medical condition/treatment analysis engine 348 analyzes the electronic medical records (EMR) of patient 302 stored in patient EMRs 322 to identify a medical condition and/or treatment associated with patient 302. Utilizing the identified medical condition and/or treatment of patient 302, correlation engine 346 identifies a portion of the patient's body that is associated with the particular medical condition and/or treatments as that part of the patient's body is viewed by user 306 via the HMD system. For example, if patient 302 has a medical condition of appendicitis, then when user 306 views the body of patient 302 through the augmented reality display of the HMD system and the lower abdomen of patient 302 comes into view, correlation engine 346 will correlate the view of the lower abdomen with the medical condition of patient 302 and provide an overlay of an appendix to be shown overlaying the lower abdomen of patient 302. Correlation engine 346 provides this overlay to display engine 350 and display engine 350 presents overlay 328 to the medical professional via the augmented reality display in the HMD system.

Of particular note to the illustrative embodiments, is that the overlay provided by correlation engine 346 may be varied depending on the view that the needs of user 306. That is, if user 306 is a nurse, then correlation engine 346 may provide a basic organ model showing a generic organ. However, if user 306 is a doctor, then correlation engine 346 may provide an actual x-ray overlay of the organ. Still further, if user 306 is a surgeon, then correlation engine 346 may provide a computerized axial tomography (CAT) scan (CT) overlay or a magnetic resonance imaging (MRI) scan overlay of the entire area. In addition to providing a basic organ model, an x-ray, CT scan, MRI scan, or the like, correlation engine 346 may also provide one or more of dissection models; overlapping organ systems; x-rays, CT scans, MRI scans, or the like, from previous medical condition/treatments; points of surgery or pressure; or the like. An indication of any additional information to provide may be identified by monitoring eye moves, facial expressions, head moved, audible utterances, or the like from the medical professional via image capture and analysis engine 342 and/or audio capture and analysis engine 344.

Additionally, the overlay provided by correlation engine 346 may be based on the time that user 306 has to spend with patient 302. For example, based on a schedule of user 306, which may be accessed by correlation engine 346 through medical professional corpus and other source data 324, user 306 may only have a few minutes to spend with patient 302 as may occur during morning rounds. Thus, correlation engine 346 may provide a basic organ model showing a generic organ. However, if the schedule shows that user 306 is performing a surgical consult prior to a surgery, then correlation engine 346 may provide an x-ray overlay, a computerized axial tomography (CAT) scan (CT) overlay, or a magnetic resonance imaging (MRI) scan overlay of the entire area. Further, whether or not the schedule shows to permits more time, if the medical professional requests, correlation engine 346 may provide any additionally overlays. That is, even though the medical professional's schedule indicates that the medical professional may only have a few minutes to spend with the patient as may occur during morning rounds and initially provide a basic organ model showing a generic organ, if the medical professional requests additional information, then correlation engine 346 may provide an x-ray overlay; a CT scan overlay; a MRI scan overlay; one or more of dissection models; overlapping organ systems; x-rays, CT scans, MRI scans, or the like, from previous medical condition/treatments; points of surgery or pressure, or the like. An indication of any additional information to provide may be identified by monitoring eye moves, facial expressions, head moved, audible utterances, or the like from the medical professional via image capture and analysis engine 342 and/or audio capture and analysis engine 344.

Still further, correlation engine 346 may provide an overlay that is based on the particular specialty of user 306 that is caring for patient 302. That is, if the identity of user 306 is an anesthesiologist or anesthetist, then correlation engine 346 may provide an organ overlay that is not even associated with the particular medical condition. That is, an anesthesiologist or anesthetist may be more concerned with the patient's lungs, airways, nasal cavities, or the like. Conversely, if the identity of user 306 is a surgeon, then correlation engine 346 may provide an organ overlay that is directly related to the particular medical condition of patient 302. Further, if the identity of user 306 is a nurse who is providing medications to patient 302, then correlation engine 346 may provide an organ overlay of where the medication is to be administered, such as a particular arm, area of an arm, or the like, of patient 302.

In addition to providing an overlay that identifies graphical images representing medical conditions, highlighting of portions of the body affected or needing to be further investigated, or the like, correlation engine 346 may also provide textual data representing lab results, treatment options, medical codes, latest medical research studies, available organs for transplant, or the like. That is, based on inputs provided by user 306, correlation engine may identify the requested textual data, which display engine 350 then displays on the augmented reality display of the HMD system. Still further, based on a mood identified using the one or more images and/or the one or more audible utterances of patient 302, correlation engine 346 may provide an indication of how user 306 should be presenting information to patient 302. That is, if correlation engine 346 identifies the mood of patient 302 as calm, then correlation engine 346 may provide an indication, which display engine 350 then displays on the augmented reality display of the HMD system, to user 306 to speak in a relaxed tone. However, if correlation engine 346 identifies the mood of patient 302 as nervous, then correlation engine 126 may provide an indication, which display engine 350 then displays on the augmented reality display of the HMD system, to user 306 to use more reassuring tones.

Additionally, once a medical professional selects, indicates, or otherwise identifies a treatment that is to be followed for the patient, which may be identified by monitoring eye moves, facial expressions, head moved, audible utterances, or the like from the medical professional, correlation engine 346 may notify one or more other medical professionals of the treatment through one or more electronic notification means, which may include scheduling a surgery, instruments to be provided during surgery, requests for a consultation, medications to be administered, or the like, in real-time, near real-time, or non-real-time. An indication of the treatment may be identified by monitoring eye moves, facial expressions, head moved, audible utterances, or the like from the medical professional via image capture and analysis engine 342 and/or audio capture and analysis engine 344.

Thus, the illustrative embodiments provide mechanisms for implementing an augmented reality display via a head mounted display (HMD) system, such as via a worn headset, glasses, or the like, that indicates the areas of a patient's body corresponding to a medical condition and/or treatment of the patient overlayed on the actual view of the patient captured by the medical professional's eyes. The mechanisms of the invention capture images of the area of the patient's body being viewed by the medical professional. Based on the part of the patient's body being viewed, the mechanisms identify the corresponding body parts in the view and correlate those body parts with the patient's electronic medical record (EMR) data indicating the medical condition and/or treatments associated with the patient.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

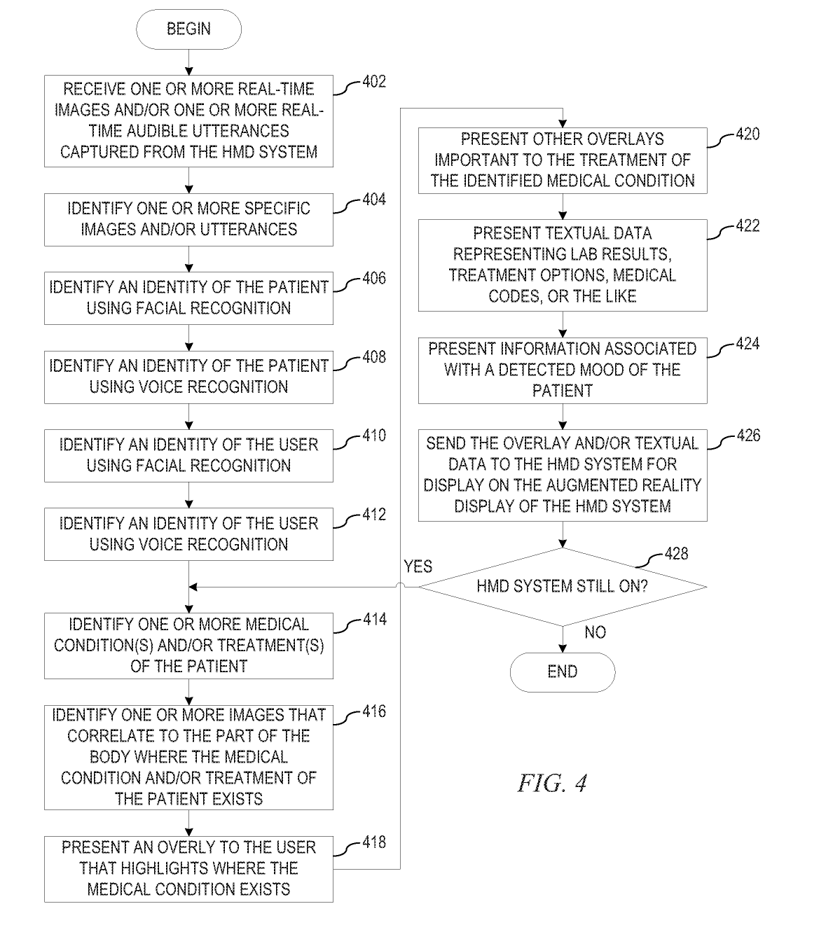

FIG. 4 depicts an exemplary flowchart of the operation performed by a cognitive healthcare system in implementing an augmented reality display via a head mounted display (HMD) system that indicates the areas of a patient's body corresponding to a medical condition and/or treatment of the patient overlayed on the actual view of the patient captured by the medical professional's eyes in accordance with an illustrative embodiment. As the operation begins, the cognitive healthcare system receives one or more real-time images and/or one or more real-time audible utterances captured from the HMD system (step 402). Utilizing the one or more real-time images and/or one or more real-time audible utterances, the cognitive healthcare system identifies one or more specific images and/or utterances (step 404), such as a facial image of the patient, a facial image of the user, an audible utterance of the patient, an audible utterance of the user, a body part of the patient, or the like. The cognitive healthcare system may identify an identity of the patient by comparing each facial image in the one or more facial images to a set of facial image stored within a set of electronic medical records (EMRs) for patients stored in corpora of patient EMRs (step 406). Alternatively or in addition to, the cognitive healthcare system may identify an identity of the patient by comparing each audible utterance in the one or more audible utterances to a set of voice recordings stored within a set of electronic medical records (EMRs) for patients stored in corpora of patient EMRs (step 408). In addition to identifying the patient, the cognitive healthcare system may identify an identity of the user by comparing each facial image in the one or more facial images to a set of facial image stored within a set of records for medical professionals stored in corpora of medical professionals (step 410). Alternatively or in addition to, the cognitive healthcare system may identify an identity of the user by comparing each audible utterance in the one or more audible utterances to a set of voice recordings stored within a set of records for medical professionals stored in corpora of medical professionals (step 412).

With the user and patient identified, the cognitive healthcare system utilizes the identity of the user to identify one or more medical condition(s) and/or treatment(s) of the patient (step 414). Utilizing the identified medical condition, the cognitive healthcare system scans the one or more body part images to identify one or more images that correlate to the part of the body where the medical condition and/or treatment of the patient exists (step 416). Then as that part of the body is being viewed by the user through the augmented reality display of the HMD system, the cognitive healthcare system presents an overly to the user that highlights where the medical condition exists (step 418). For example, the cognitive healthcare system may present a basic organ model showing a generic organ, an actual x-ray overlay of the organ, a computerized axial tomography (CAT) scan (CT) overlay or a magnetic resonance imaging (MRI) scan overlay of the entire area, or the like. The overlay that is utilized by the cognitive healthcare system may be based on the level or specialty of the user, may be based on a schedule associated with the user, or the like.

In addition to providing the overlay associated with the identified medical condition and/or treatment, the cognitive healthcare system may present other overlays (step 420) that are not particular to the medical condition but may be important to the treatment of the identified medical condition, such as providing an overly showing the patient's lungs, airways, nasal cavities, or the like, to an anesthesiologist or anesthetist who may be involved with an upcoming surgery, or an overlay of where a particular medication is to be administered to a nurse who is caring for the patient. Further, the cognitive healthcare system may present textual data representing lab results, treatment options, medical codes, or the like (step 422). Still further, the cognitive healthcare system may present information associated with a detected mood of the patient (step 424) in order that the user may change his or her tone when speaking with the patient. Regardless of the overlay and/or textual data that is identified to be presented, cognitive healthcare system sends the overlay and/or textual data to the HMD system for display on the augmented reality display of the HMD system (step 426). The cognitive healthcare system then determined whether the HMD system has been turned off (step 428). If at step 428 the HMD system has not been turned off, the process returns to 414 since the overlays and/or textual data may need to change overtime as the user interacts with the patient. If at step 428 the HMD system is turned off, the operation ends.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide mechanisms for implementing an augmented reality display via a head mounted display (HMD) system, such as via a worn headset, glasses, or the like, that indicates the areas of a patient's body corresponding to a medical condition and/or treatment of the patient overlayed on the actual view of the patient captured by the medical professional's eyes. The mechanisms of the invention capture images of the area of the patient's body being viewed by the medical professional. Based on the part of the patient's body being viewed, the mechanisms identify the corresponding body parts in the view and correlate those body parts with the patient's electronic medical record (EMR) data indicating the medical condition and/or treatments associated with the patient. In some cases, facial recognition may be utilized to identify the particular patient being viewed.

The superimposed graphical representations on the patient's body may be medical condition and implementation specific. That is, the superimposed graphical representations may include graphical images representing medical conditions, highlighting of portions of the body affected or needing to be further investigated, textual data representing lab results, treatment options, medical codes, or the like. The mechanisms also provide access to a medical corpus of data annotated for multiple media views to allow the real time selection of media suitable for a given patient mood, the time of the day, the medical professional's schedule availability, or the like. With regard to the medical professional's schedule, depending on the schedule, a more compressed (basic organ model) may be displayed with the availability is limited, whereas a detailed (surgery technique simulated on the patient organ) may be displayed when the availability is extended.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

1. A method, in a data processing system comprising at least one processor and at least one memory, the at least one memory comprising instructions executed by the at least one processor to cause the at least one processor to implement a cognitive healthcare system, wherein the cognitive healthcare system operates to:
capturing, by a capturing mechanism of the cognitive healthcare system, a real-time image of an area of a patient's body being viewed by a medical professional via a head mounted display (HMD) system;
identifying, by the cognitive healthcare system, one or more body parts of the patient within the real-time image;
correlating, by the cognitive healthcare system, the one or more identified body parts with the patient's electronic medical records (EMRs) indicating the medical condition and/or treatments associated with the patient; and
generating, by the cognitive healthcare system, an augmented reality display, in the HMD system, of one or more areas of the patient's body corresponding to the medical condition and/or treatment of the patient overlaying the real-time image of the area of the patient's body, wherein a level of information displayed in the augmented reality display is based on a schedule of the medical professional such that the cognitive healthcare system:
accesses a schedule of the medical professional through a medical professional corpus or corpora of data;
determines an amount of time the medical professional has to spend with the patient; and
displays the level of information in the augmented reality display commensurate with the amount of time the medical professional has to spend with the patient.

2. The method of claim 1, wherein the patient's electronic medical records (EMRs) are correlated to the patient by the capturing mechanism capturing an image of the patient's face and the cognitive healthcare system utilizing facial recognition to identify the patient.

3. The method of claim 1, wherein the patient's electronic medical records (EMRs) are correlated to the patient by the capturing mechanism capturing an audible utterance from the patient and the cognitive healthcare system utilizing voice recognition to identify the patient.

4. The method of claim 1, wherein the augmented reality display displays one or more of a basic organ model, a current x-ray, a current computerized axial tomography (CAT) scan (CT), a current magnetic resonance imaging (MRI) scan, one or more of dissection models, overlapping organ systems, previous x-rays, previous CT scans, previous MRI scans, or points of surgery or pressure.

5. The method of claim 1, wherein the augmented reality display further highlights portions of the patients' body affecting or needing to be further investigated with regard to the medical condition.

6. The method of claim 1, wherein the augmented reality display further displays textual data representing lab results, treatment options, medical codes, latest medical research studies, or available organs for transplant.

7. The method of claim 1, wherein the cognitive healthcare system further:
captures a facial expression of the patient;
captures one or more audible utterances of the patient;
identifies a mood of the patient using the captured facial expression and the one or more audible utterances; and
displays via the augmented reality display an indication of how the medical professional should be presenting information to the patient mood.

8. The method of claim 1, wherein the medical professional treating the patient is identified by the capturing mechanism capturing an image of the medical professional's face and the cognitive healthcare system utilizing facial recognition to identify the medical professional.

9. The method of claim 1, wherein the medical professional treating the patient is identified by the capturing mechanism capturing an audible utterance from the medical professional and the cognitive healthcare system utilizing voice recognition to identify the medical professional.

10. (canceled)

11. A computer program product comprising a computer readable storage medium having a computer readable program stored therein, wherein the computer readable program, when executed on a computing device, causes the computing device to:
capture, by a capturing mechanism, a real-time image of an area of a patient's body being viewed by a medical professional via a head mounted display (HMD) system;
identify one or more body parts of the patient within the real-time image;
correlate the one or more identified body parts with the patient's electronic medical records (EMRs) indicating the medical condition and/or treatments associated with the patient; and
generate an augmented reality display, in the HMD system, of one or more areas of the patient's body corresponding to the medical condition and/or treatment of the patient overlaying the real-time image of the area of the patient's body, wherein a level of information displayed in the augmented reality display is based on a schedule of the medical professional such that the computer readable program causes the computing device to:
access a schedule of the medical professional through a medical professional corpus or corpora of data;
determine an amount of time the medical professional has to spend with the patient; and
display the level of information in the augmented reality display commensurate with the amount of time the medical professional has to spend with the patient.

12. The computer program product of claim 11, wherein the augmented reality display displays one or more of a basic organ model, a current x-ray, a current computerized axial tomography (CAT) scan (CT), a current magnetic resonance imaging (MRI) scan, one or more of dissection models, overlapping organ systems, previous x-rays, previous CT scans, previous MRI scans, or points of surgery or pressure.

13. The computer program product of claim 11, wherein the augmented reality display further highlights portions of the patients' body affecting or needing to be further investigated with regard to the medical condition.

14. The computer program product of claim 11, wherein the augmented reality display further displays textual data representing lab results, treatment options, medical codes, latest medical research studies, or available organs for transplant.

15. The computer program product of claim 11, wherein the computer readable program further causes the computing device to:
capture a facial expression of the patient;
capture one or more audible utterances of the patient;
identify a mood of the patient using the captured facial expression and the one or more audible utterances; and
display via the augmented reality display an indication of how the medical professional should be presenting information to the patient mood.

16. An apparatus comprising:
a processor; and
a memory coupled to the processor, wherein the memory comprises instructions which, when executed by the processor, cause the processor to:
capture, by a capturing mechanism, a real-time image of an area of a patient's body being viewed by a medical professional via a head mounted display (HMD) system;
identify one or more body parts of the patient within the real-time image;
correlate the one or more identified body parts with the patient's electronic medical records (EMRs) indicating the medical condition and/or treatments associated with the patient; and
generate an augmented reality display, in the HMD system, of one or more areas of the patient's body corresponding to the medical condition and/or treatment of the patient overlaying the real-time image of the area of the patient's body, wherein a level of information displayed in the augmented reality display is based on a schedule of the medical professional such that the instructions cause the processor to:
access a schedule of the medical professional through a medical professional corpus or corpora of data;
determine an amount of time the medical professional has to spend with the patient; and
display the level of information in the augmented reality display commensurate with the amount of time the medical professional has to spend with the patient.

17. The apparatus of claim 16, wherein the augmented reality display displays one or more of a basic organ model, a current x-ray, a current computerized axial tomography (CAT) scan (CT), a current magnetic resonance imaging (MRI) scan, one or more of dissection models, overlapping organ systems, previous x-rays, previous CT scans, previous MRI scans, or points of surgery or pressure.

18. The apparatus of claim 16, wherein the augmented reality display further highlights portions of the patients' body affecting or needing to be further investigated with regard to the medical condition.

19. The apparatus of claim 16, wherein the augmented reality display further displays textual data representing lab results, treatment options, medical codes, latest medical research studies, or available organs for transplant.

20. The apparatus of claim 16, wherein the instructions further cause the processor to:
capture a facial expression of the patient;
capture one or more audible utterances of the patient;
identify a mood of the patient using the captured facial expression and the one or more audible utterances; and
display via the augmented reality display an indication of how the medical professional should be presenting information to the patient mood.
